(19) United States
(12) Patent Application Publication
(10) Pub. No.: US 2026/0077147 A1
(43) Pub. Date: Mar. 19, 2026

(54) CPAP MOUTHPIECE AND NOSE PILLOW FOR HEADBAND-FREE USE

(71) Applicant: Dennis Comer, Los Angeles, CA (US)

(72) Inventor: Dennis Comer, Los Angeles, CA (US)

(21) Appl. No.: 18/888,956

(22) Filed: Sep. 18, 2024

(51) Int. Cl. A61M 16/06

(52) U.S. Cl. CPC A61M 16/0622; A61M 16/0688

(57) ABSTRACT

The invention is a mouth and nose interface subsystem operative to attach to the positive-pressure air provided by a CPAP machine, and to be attached to the mouth and nose, respectively, using facial-skin adhesive strips rather than headbands that encircle the head.

### TECHNICAL FIELD

The invention is a mouthpiece and nose pillow subsystem for use with a continuous-positive-airway-pressure (CPAP) system.

### BACKGROUND OF INVENTION

It is estimated that 30 million US residents suffer from sleep apnea whereas only six million have thus far been diagnosed.

Those who have sleep apnea are often plagued with waking-time fatigue due to interrupted sleep patterns. It is also considered a risk factor for heart attacks, strokes and higher likelihood of driving accidents.

Those who are diagnosed with sleep apnea are often prescribed a CPAP system to be used nightly while sleeping. By providing a pressurized stream of air through mouth and/or nose, one's airway is kept open and sleep apnea symptoms (e.g. periods of interrupted air intake) abate.

To prevent dislodgement of nose and/or mouth interfaces (e.g. mouthpiece and/or nose pillow), these CPAP subsystems are typically held in place by headbands that encircle the user's head and resist dislodgement during change in position during sleep. In some cases, the mouthpiece and nose pillow are replaced with a mask that covers both nose and mouth and is also held in place with headbands.

Many users find the headbands uncomfortable and may forego using the CPAP system on a regular basis.

### BRIEF DESCRIPTION OF INVENTION

The invention comprises a mouthpiece subsystem and a nose pillow subsystem that are operative to be held in position and resist dislodgement using a replaceable adhesive strip on each side that adheres to the user's facial skin without causing skin irritation or off-putting discomfort.

The mouthpiece subsystem is designed such that positive air pressure may be confined to just the mouthpiece when it is used alone, or distributed to both mouthpiece and nose pillow subsystems when used together.

The replaceable adhesive strip subsystems are operative to anchor their mouthpiece and nose pillow subsystems, resisting dislodgement, without requiring head-encircling headbands.

### BRIEF DESCRIPTION OF DRAWINGS

FIG. 1 depicts a contemporary CPAP system, with headbands, applied to a sleeping person.

FIG. 2 illustrates the structures of the invention's mouthpiece subsystem.

FIG. 3 illustrates the structures of the invention's nose pillow subsystem.

FIG. 4 shows how the invention is used by a user wishing only to have a mouthpiece interface.

FIG. 5 shows how the invention is used by a user wishing to have both a mouthpiece and nose pillow interface.

### DETAILED DESCRIPTION OF INVENTION

Since the CPAP system was first introduced in 1980, subsequent innovations have made them smaller, quieter, and more convenient to use. But, some users balk at the discomfort of sleeping with mouth and/or nose interfaces being held in place by headbands that encircle the head while trying to fall asleep.

The invention herein disclosed uses the standard CPAP machine and air-distribution hose but introduces a compatible mouth and nose interface that does not require a headband to be held in place.

What's more, the way the mouthpiece is designed, it can be used alone, as the sole interface, or in conjunction with a nose pillow.

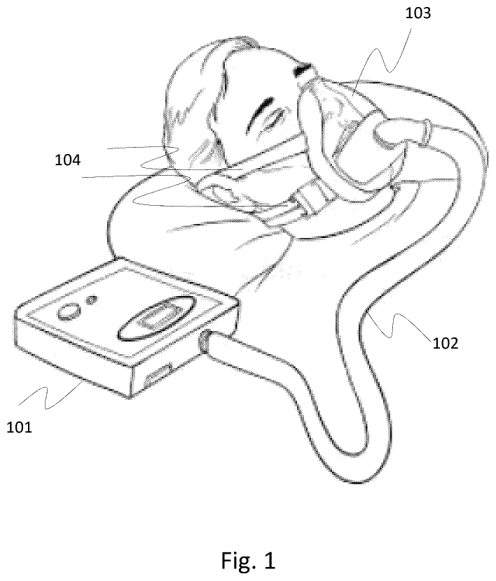

FIG. 1 shows a contemporary CPAP system in use by a person with sleep apnea. The machine that applies the continuous positive airway pressure (101) is connected by a flexible hose (102) to a face mask (103) held in place by a pair of head encircling headbands (104). Many users find the headbands uncomfortable and may avoid daily use of the system as a result.

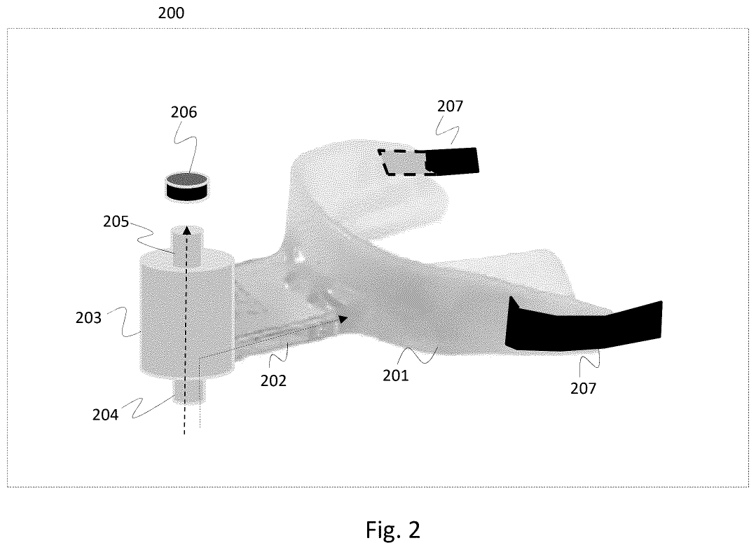

In FIG. 2, a mouthpiece subsystem (200) comprises the in-the-mouth portion (201), the air-pressure bridge portion (202), the port structure (203) with CPAP system compatible air-intake port (204) and air-exit port (205). When used as the sole interface, a covering structure (206) keeps the air flow confined to the mouthpiece subsystem. And, attached to the sides of the in-the-mouth portion is a pair of replaceable, rectangular-shaped, adhesive strips (207).

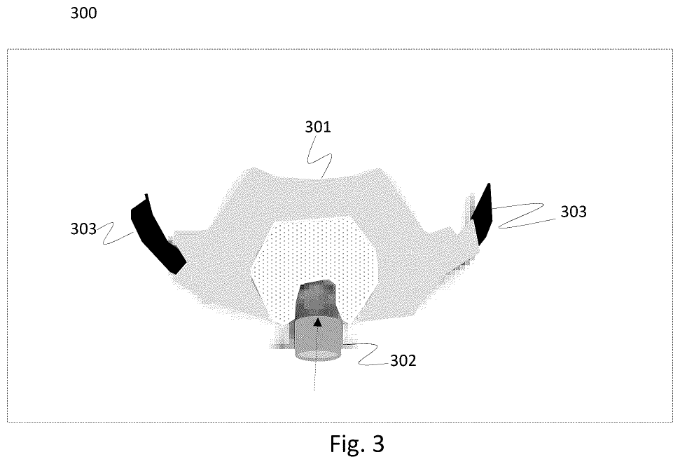

In FIG. 3, a nose pillow subsystem (300) comprises the external nose interface portion (301), the CPAP system compatible air-intake port (302) and replaceable adhesive strips (303). When the nose pillow and mouthpiece are used, a small section of hose joins the mouthpiece's air-exit port (FIG. 2, 205) to the nose pillow's input port (302). When so joined, the air flow is distributed to both the mouthpiece and nose pillow interfaces.

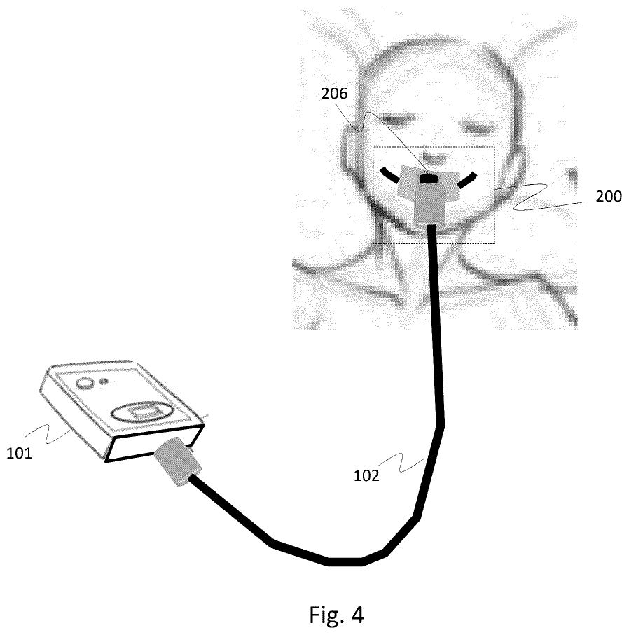

FIG. 4 shows the CPAP system configured for sole use of the mouthpiece (200) wherein the CPAP machine (101) conveys positive-pressure airflow via hose 102, to the mouthpiece air-input port and the air-exit port is capped by the covering structure (206).

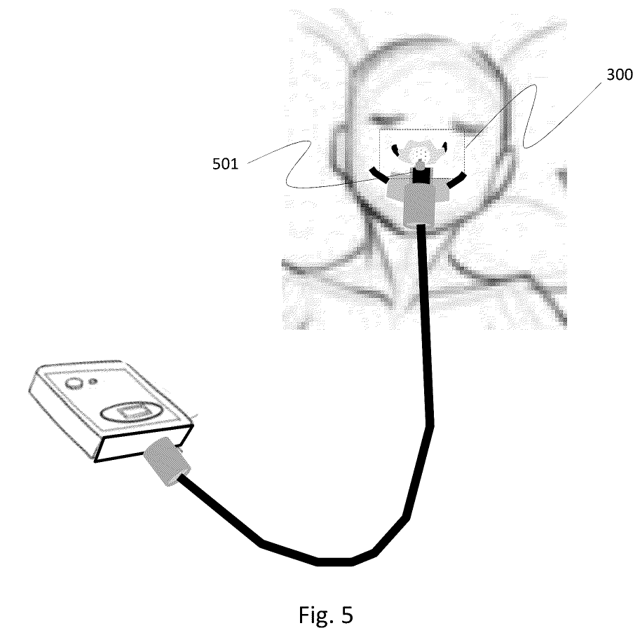

FIG. 5 shows the system configured for using a combination of mouthpiece and nose pillow (300). Here, the nose pillow is connected to the mouthpiece's exit port by a short section of hose (501).

The mouthpiece and nose pillow can be made of non-toxic, flexible material. For example, the air intake and exit port and air bridge portion can be more rigid plastic whereas the in-the-mouth portion can be more flexible silicone materials. It could be molded as a single casting or separately cast and firmly attachable.

The adhesive can be made of water-based acrylic adhesives which are non-toxic, provide a strong bond for long periods without irritation, are highly water resistant and dry clear. In addition, they are able to bond to flexible, plastic strips.

The drawings and descriptions are exemplary and should not be read as limiting the scope of claims.

What is claimed is:

1. A mouth and a nose CPAP interface subsystem comprising:
a mouthpiece comprising:
an in-the-mouth portion operative to conform to different mouth size and shape;
an air-intake and an air-exit port structure joined to the in-the-mouth portion through an air bridge through which positive pressure airflow is conveyed to the in-the-mouth portion and to a user's mouth;
the air-intake port is operative to connect to a hose conveying positive-pressure air flow;
the air-exit port is operative to connect to a short-hose structure conveying air to a nose-pillow interface subsystem;
the nose-pillow interface system operative to convey positive-pressure air flow to a nose;
the nose-pillow is operative to connect to the short-hose structure conveying positive-pressure air flow from the air-exit port;
a first pair of flexible, rectangular strips, each one attached to opposite sides of the in-the-mouth portion;
a second pair of flexible, rectangular strips, each one attached to opposite side of the nose-pillow interface;
the first pair and the second pair of flexible, rectangular strips are coated on one side by a non-toxic, acrylic adhesive operative to adhere firmly to facial skin without causing skin irritation.

2. A claim as in claim 1 wherein:
the first pair and the second pair of flexible, rectangular strips are detachable and replaceable.

3. A method claim comprising:
connecting a mouthpiece subsystem to a hose that provides a positive-pressure air flow;
connecting a covering structure to an air-exit port on the mouthpiece subsystem;
placing an in-the-mouth portion of the mouthpiece subsystem into a mouth; and
pressing each of two flexible, rectangular strips, coated with acrylic adhesive, to facial skin on opposite sides of a mouth, so as to adhere to the skin, and resist dislodgement.

4. A method claim comprising:
connecting the mouthpiece subsystem to the hose that provides a positive-pressure air flow;
connecting a short section of hose to the air-exit port on the mouthpiece subsystem;
connecting the opposite end of the short section of hose to a nose-pillow interface's air intake port;
placing the in-the-mouth portion of the mouthpiece subsystem into a mouth;
placing the nose-pillow interface over a nose;
pressing each of the two flexible, rectangular strips, coated with acrylic adhesive, attached to the mouthpiece subsystem to facial skin on opposite sides of a mouth, so as to adhere to the skin, and resist dislodgement; and
pressing each of two flexible, rectangular strips, coated with acrylic adhesive, attached to the nose-pillow subsystem to facial skin on opposite sides of a nose, so as to adhere to the skin and resist dislodgement.
